(19) United States
(12) Patent Application Publication
(10) Pub. No.: US 2020/0391181 A1
(43) Pub. Date: Dec. 17, 2020

(54) LIQUID ABSORBENT BODY

(71) Applicant: SEIKO EPSON CORPORATION, Tokyo (JP)

(72) Inventor: Yoichi MIYASAKA, Suwa (JP)

(21) Appl. No.: 16/898,521

(22) Filed: Jun. 11, 2020

(30) Foreign Application Priority Data
Jun. 13, 2019 (JP) 2019-110149

(51) Int. Cl. B01J 20/24; B01J 20/26; B01J 20/28; C12M 1/00; B32B 5/02; B32B 27/12; B32B 27/06; B32B 27/30; B32B 29/00; B32B 29/02

(52) U.S. Cl. CPC B01J 20/24; B01J 20/26; B01J 20/28038; B01J 20/28035; C12M 29/00; B32B 5/02; B32B 2307/726; B32B 27/06; B32B 27/308; B32B 29/002; B32B 29/02; B32B 2262/062; B32B 27/12

(57) ABSTRACT

A liquid absorbent body includes a water absorbent resin, and a small piece aggregate that includes a plurality of small pieces having a pulp fiber base material carrying the water absorbent resin, in which the pulp fiber base material is provided with a cutout portion that exposes a pulp fiber inside the pulp fiber base material, other than an outer peripheral end surface that defines an outer shape of the small piece. The cutout portion is preferably provided at a depth of half or more of the pulp fiber base material in a thickness direction. The small piece preferably includes a pair of pulp fiber base materials, and the water absorbent resin is preferably provided between the pulp fiber base materials.

The present application is based on, and claims priority from JP Application Serial Number 2019-110149, filed Jun. 13, 2019, the disclosure of which is hereby incorporated by reference herein in its entirety.

### BACKGROUND

### 1. Technical Field

The present disclosure relates to a liquid absorbent body.

### 2. Related Art

In an ink jet printer, a head cleaning operation is normally performed to prevent a decrease in print quality due to clogging of ink. Waste ink is generated during the head cleaning operation and an ink filling operation after an ink cartridge is replaced. Therefore, in order to prevent such waste ink from being attached unintentionally to a mechanism or the like inside the printer, a liquid absorbent body that absorbs the waste ink is provided.

In the related art, as a liquid absorbent body, a liquid absorbent-block containing a hydrophilic fiber and a superabsorbent polymer is used (for example, refer to JP-A-4-90851).

However, the above liquid absorbent body is formed in a block shape as a whole, does not follow a container, and it is difficult to control the amount and density of the liquid absorbent body in the container.

Furthermore, in the above liquid absorbent body, although the density was increased in order to maximize an effect of capillary phenomenon and increase the amount of water retention, there was a problem that a permeation rate at an upper surface of an absorbent body is reduced, and a pigment and the like of an ink component is coated and accumulated to inhibit the permeation to an end of the container. Although an effect of accumulation of the ink component was reduced by hollowing a portion of the absorbent body to provide an accumulation space, the amount of absorption decreases by the amount of hollowing, and as a result, there was a problem that the container size had to be increased.

### SUMMARY

The present disclosure can be realized in the following application examples.

A liquid absorbent body according to an application example of the present disclosure includes a water absorbent resin, and a small piece aggregate that includes a plurality of small pieces having a pulp fiber base material carrying the water absorbent resin, in which the pulp fiber base material is provided with a cutout portion that exposes a pulp fiber inside the pulp fiber base material, other than an outer peripheral end surface that defines an outer shape of the small piece.

### BRIEF DESCRIPTION OF THE DRAWINGS

FIG. 1 is a perspective view illustrating an example of a small piece provided in a liquid absorbent body.

FIG. 2 is a perspective view illustrating an example of a small piece provided in the liquid absorbent body.

FIG. 3 is a perspective view illustrating an example of a small piece provided in the liquid absorbent body.

FIG. 4 is a perspective view illustrating an example of a small piece provided in the liquid absorbent body.

FIG. 5 is a perspective view illustrating an example of a small piece provided in the liquid absorbent body.

FIG. 6 is a perspective view illustrating an example of a small piece provided in the liquid absorbent body.

FIG. 7 is a view illustrating an example of a method of forming a cutout portion having an aspect illustrated in FIG. 4.

FIG. 8 is a partial cross-sectional view illustrating an example of a small piece provided in the liquid absorbent body.

FIG. 9 is a partial cross-sectional view illustrating another example of a small piece provided in the liquid absorbent body.

FIG. 10 is a diagram schematically illustrating a state where a small piece having no cutout portion absorbs a liquid applied from one main surface side of the small piece.

FIG. 11 is a diagram schematically illustrating a state where a small piece having a cutout portion absorbs the liquid applied from one main surface side of the small piece.

FIG. 12 is a partial vertical cross-sectional view illustrating an example of an ink absorber as a liquid absorber using the liquid absorbent body as an ink absorbent material, and an example of a printing apparatus as a liquid treatment apparatus.

### DESCRIPTION OF EXEMPLARY EMBODIMENTS

Hereinafter, preferred embodiments of the present disclosure will be described in detail.

### 1. Liquid Absorbent Body

First, a liquid absorbent body of the present disclosure will be described.

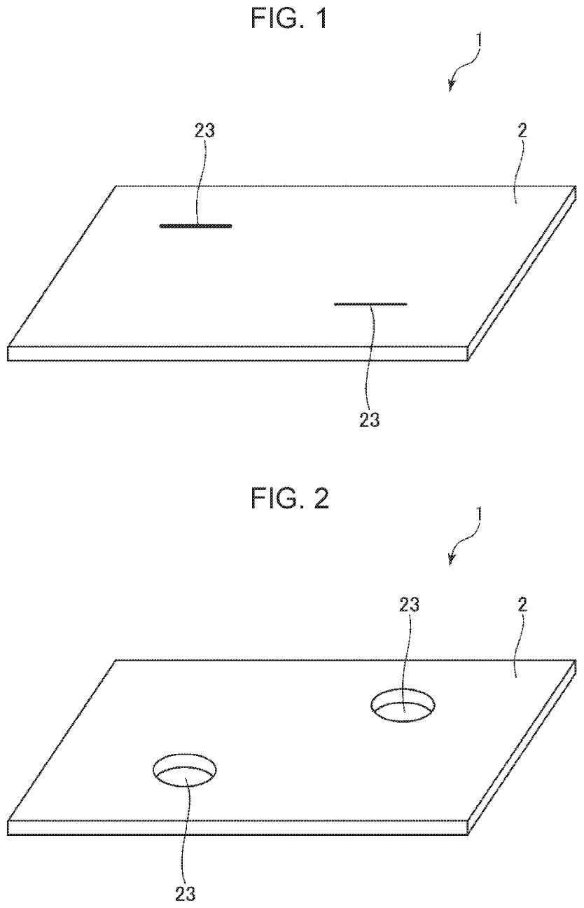

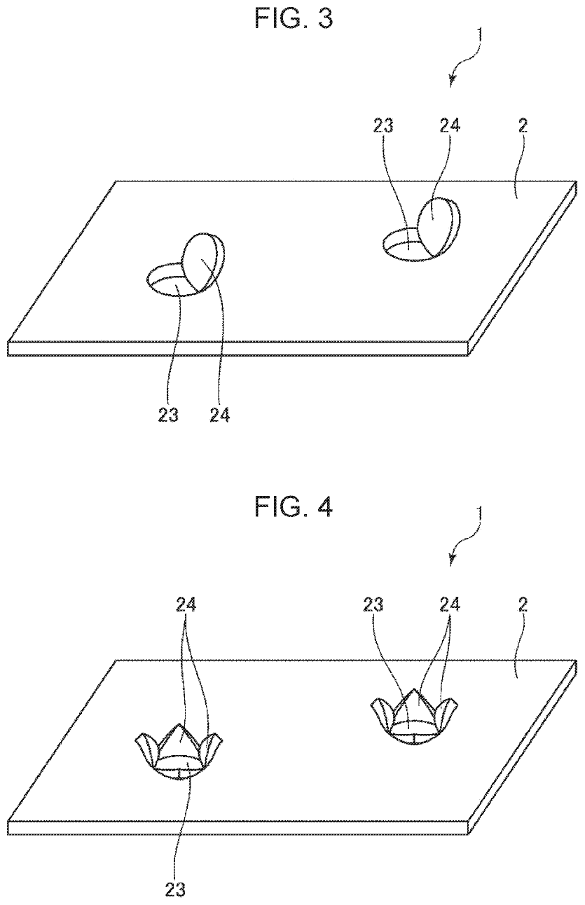

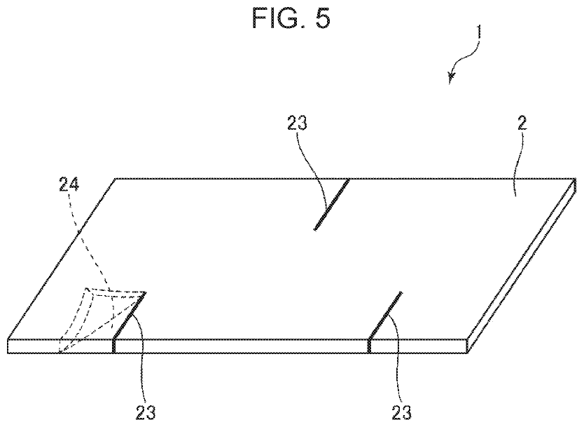

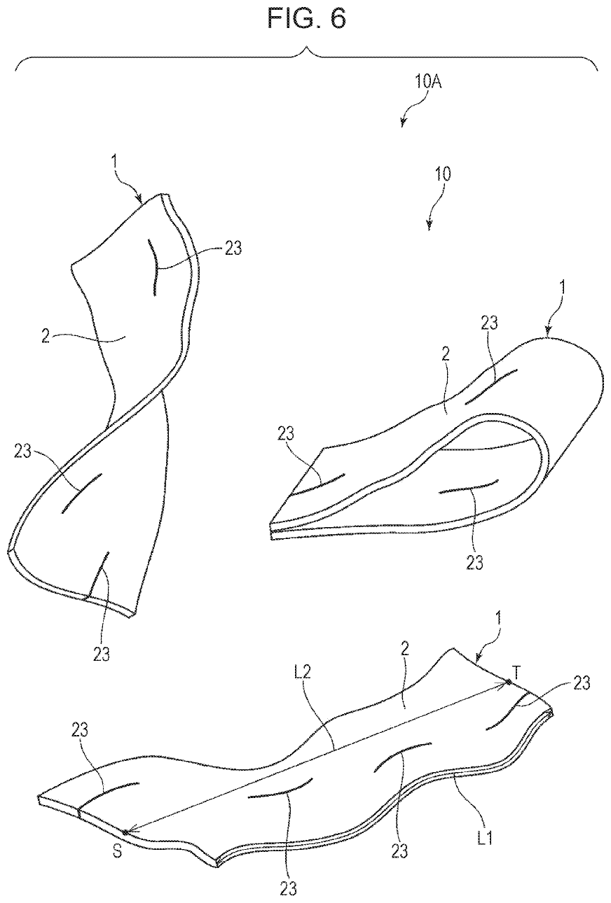

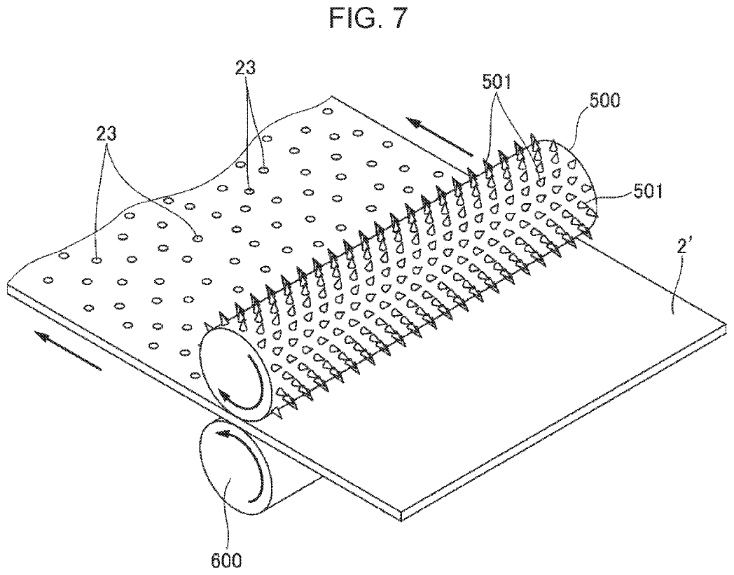

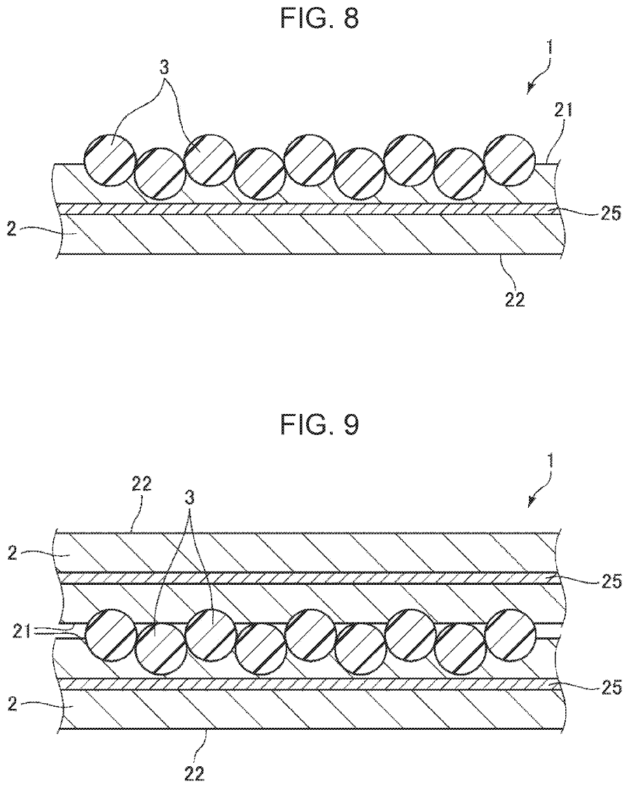

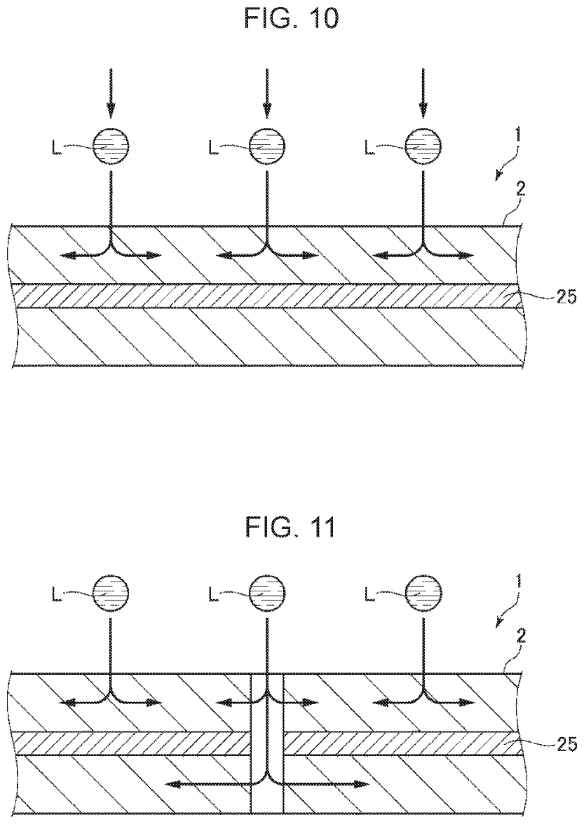

FIG. 1 is a perspective view illustrating an example of a small piece provided in a liquid absorbent body. FIG. 2 is a perspective view illustrating an example of a small piece provided in the liquid absorbent body. FIG. 3 is a perspective view illustrating an example of a small piece provided in the liquid absorbent body. FIG. 4 is a perspective view illustrating an example of a small piece provided in the liquid absorbent body. FIG. 5 is a perspective view illustrating an example of a small piece provided in the liquid absorbent body. FIG. 6 is a perspective view illustrating an example of a small piece provided in the liquid absorbent body. FIG. 7 is a diagram illustrating an example of a method of forming a cutout portion having an aspect illustrated in FIG. 4. FIG. 8 is a partial cross-sectional view illustrating an example of a small piece provided in the liquid absorbent body. FIG. 9 is a partial cross-sectional view illustrating another example of a small piece provided in the liquid absorbent body. FIG. 10 is a diagram schematically illustrating a state where a small piece having no cutout portion absorbs a liquid applied from one main surface side of the small piece. FIG. 11 is a view schematically illustrating a state where a small piece having a cutout portion absorbs the liquid applied from one main surface side of the small piece. In FIGS. 1 to 6, 10 and 11, illustrations of the water absorbent resin 3 are omitted. In addition, FIGS. 8 and 9 are enlarged views illustrating a portion of the small piece 1, in which a cutout portion 23 is not provided.

Hereinafter, for convenience of description, upper sides in FIGS. 1 to 11 are referred to as “upper” or “upward”, and lower sides are referred to as “lower” or “downward”.

The liquid absorbent body 10A is configured to include a water absorbent resin 3 and a small piece aggregate 10 including a plurality of small pieces 1 having a pulp fiber base material 2 carrying the water absorbent resin 3. As illustrated in FIGS. 1 to 6, the pulp fiber base material 2 constituting the small piece 1 is provided with a cutout portion 23 that exposes the pulp fiber inside the pulp fiber base material 2, other than an outer peripheral end surface that defines an outer shape of the small piece 1.

The liquid absorbent body 10A is normally used by filling a predetermined container such as a container 7 in an ink absorber 100 as described later.

Since the liquid absorbent body 10A is not formed into a predetermined shape in advance such as a block shape, and is an aggregate of the small pieces 1, for example, when filling a container such as the container 7 described later, the relative positional relationship between the small pieces 1 can be changed. That is, the shape of the small piece aggregate 10 can be easily changed. Therefore, the small piece aggregate 10 changes the shape of the liquid absorbent body 10A as a whole according to the shape of the storage space of the container. Therefore, in the container, it is possible to effectively prevent occurrence of unintentional variations in density. In particular, not only for containers of specific shapes and sizes, containers of various shapes can be filled at a desired density while effectively preventing the occurrence of unintentional variations in density. As a result, the absorption efficiency of the water-containing liquid can be made excellent in the liquid absorbent body 10A in a state of being stored in the container.

In addition, the cutout portion 23 that exposes the pulp fiber inside the pulp fiber base material 2 is provided at a portion other than the outer peripheral end surface of the pulp fiber base material 2 that defines the outer shape of the small piece 1. Therefore, the cutout portion 23 can also absorb the liquid in addition to the both main surfaces and the end surfaces of the small piece 1, and the absorption rate of the liquid of the liquid absorbent body 10A as a whole can be made excellent. In particular, in the cutout portion 23, normally, the capillary phenomenon is effectively exerted, so that the absorption rate of the liquid can be made more excellent.

On the other hand, when the above conditions are not satisfied, the above good effects cannot be obtained.

For example, when the liquid absorbent body is formed in a predetermined shape such as a block shape, there is no followability to the container, and it is difficult to adjust the amount of the liquid absorbent body filled in the container according to the size and shape of the container. In addition, unintended unevenness is likely to occur in the liquid absorption characteristics at each portion in the container.

In addition, even when the liquid absorbent body is configured to include the water absorbent resin and the aggregate of small pieces having a pulp fiber base material carrying the water absorbent resin, if the small pieces are not provided with the cutout portion, it is difficult to sufficiently increase the liquid absorption efficiency.

In addition, even when the liquid absorbent body includes the water absorbent resin and the pulp fiber base material that carries the water absorbent resin, is in the form of a sheet, and is provided with the cutout portion in the pulp fiber base material, when the liquid absorbent body is not the aggregate of small pieces, the followability to the container is low, and it is difficult to adjust the amount of the liquid absorbent body filled in the container according to the size and shape of the liquid absorbent body. In addition, unintended unevenness is likely to occur in the liquid absorption characteristics at each portion in the container.

In the present disclosure, the external shape refers to a contour shape of a small piece in which the cutout portion is not formed when the small piece is viewed in plan view from a direction in which the projected area is largest.

Hereinafter, a configuration of the small piece 1 constituting the liquid absorbent body 10A will be described.

The small piece 1 includes the pulp fiber base material 2 containing the pulp fiber and the water absorbent resin 3 carried on the pulp fiber base material 2. More specifically, the liquid absorbent body 10A is configured to include the small piece aggregate 10 that is an aggregate of the plurality of small pieces 1 configured such that the pulp fiber base material 2 containing the pulp fiber carries the water absorbent resin 3.

In the present specification, “water absorption” refers to absorbing water itself or a liquid containing water such as ink or body fluids. In addition, in the present specification, “liquid” refers to water itself or a liquid containing water such as ink or body fluids, unless otherwise specified. In particular, examples of a preferable liquid include a liquid containing water at a content of 50% by mass or more.

It is preferable that each small piece 1 has flexibility and has a longitudinal shape. Here, a longitudinal shape refers to a shape having an aspect ratio of 1.5 or more, and corresponds to a so-called strip shape, a shredder piece, or the like.

Each of the small pieces 1 having such a shape is easily deformed. In particular, when the liquid absorbent body 10A is stored in the container, each of the small pieces 1 is deformed regardless of the shape of the inside of the container, that is, the shape followability is more effectively exhibited. Therefore, the liquid absorbent body 10A is stored in a lump without difficulty. In addition, the contact area of the liquid absorbent body 10A as a whole with the liquid can be ensured as much as possible, and therefore, the absorption characteristics for absorbing the liquid are improved.

As described above, the pulp fiber base material 2 is provided with the cutout portion 23 other than the outer peripheral end surface that defines the outer shape of the small piece 1.

The cutout portion 23 may be any portion that exposes the pulp fiber inside the pulp fiber base material 2, and is preferably provided at a depth of at least half or more of the pulp fiber base material 2 in the thickness direction.

As a result, the area of the portion where the pulp fiber is exposed in the cutout portion 23 can be made larger, and the absorption rate of the liquid of the entire liquid absorbent body 10A can be made more excellent.

In addition, when the pulp fiber base material 2 includes a sizing agent layer 25 described later, it is preferable that the cutout portion 23 is provided to penetrate the sizing agent layer 25.

In the configurations illustrated in FIGS. 1, 5, and 6, the cutout portion 23 is provided as a slit that penetrates the pulp fiber base material 2 in the thickness direction. As a result, the area of the portion where the pulp fiber is exposed in the cutout portion 23 can be further increased. In addition, even when the liquid enters from any of the main surface directions of the small pieces 1, the cutout portion 23 can efficiently absorb the liquid. As described above, the absorption rate of the liquid of the entire liquid absorbent body 10A can be made particularly excellent. In addition, since a portion of the small piece 1 is not removed by the cutout portion 23, the weight of the material capable of absorbing the liquid can be increased, which is advantageous in increasing the amount of the liquid that can be absorbed by the liquid absorbent body 10A.

The slit as the cutout portion 23 illustrated in FIG. 1 is inside the small piece 1 in plan view and does not reach the outer periphery of the small piece 1. The slit illustrated as the cutout portion 23 in FIG. 5 reaches the outer periphery of the small piece 1 in plan view.

The shape of the slit may be a straight line shape, a broken line shape, or a shape having a curved line such as, for example, an arc shape or an S shape in plan view, as illustrated in FIGS. 1 and 5. In addition, the slit may be a cross cut where a plurality of lines intersect.

In addition, in the configurations illustrated in FIGS. 2, 3, and 4, the cutout portion 23 is provided as a through-hole that penetrates the pulp fiber base material 2 in the thickness direction. As a result, the area of the portion where the pulp fiber is exposed in the cutout portion 23 can be further increased, and the pulp fiber inside the pulp fiber base material 2 can be more efficiently exposed. In addition, even when the liquid enters from any of the main surface directions of the small pieces 1, the cutout portion 23 can efficiently absorb the liquid. As described above, the absorption rate of the liquid of the entire liquid absorbent body 10A can be made particularly excellent.

The shape of the through-hole may be circular, oval, polygonal, or the like in plan view, as illustrated in FIGS. 2, 3, and 4.

In the configurations illustrated in FIGS. 3 and 4, the material in the through-hole rises by the cutout portion 23 as a through-hole penetrating the pulp fiber base material 2 in the thickness direction to form a cutout piece 24. The cutout piece 24 is coupled to a periphery of the cutout portion 23 which is a through-hole at a base portion. In other words, the through-hole is provided in a state where the material of the pulp fiber base material 2 for the size of the through-hole is not detached from the pulp fiber base material 2 and at least a portion thereof is left. Furthermore, in other words, at least a portion of the material of the pulp fiber base material 2 removed by the formation of the through-hole remains in the vicinity of the through-hole.

As a result, the raised cutout pieces 24 can function as spacers for securing a gap between the small pieces 1, and a bulk density of the liquid absorbent body 10A can be adjusted more easily. In addition, when the gap is small, the liquid permeability of the liquid can be further improved by a capillary phenomenon. As a result, the liquid flowing downward in the container is more effectively prevented from being blocked in the middle, and thus can more suitably permeate into the bottom portion of the container. As a result, each of the small pieces 1 can suitably absorb the liquid and hold the liquid.

In the configuration illustrated in FIG. 5, the cutout piece 24 is provided which is formed by the cutout portion 23 as a slit penetrating the pulp fiber base material 2 in the thickness direction. The cutout piece 24 is coupled to the periphery of the cutout portion 23 which is a slit at the base portion.

Even in such a case, the effect of having the cutout pieces 24 as described above is exerted.

The cutout portion 23 illustrated in FIG. 3 can be said to be a through-hole and also an arc-shaped slit.

In the configurations illustrated in FIGS. 5 and 6, the cutout portion 23 is coupled to the outer shape of the pulp fiber base material 2 of the small piece 1. As a result, when a stress such as twisting or bending is applied to the pulp fiber base material 2, the pulp fiber base material 2 in a portion adjacent to the cutout portion 23 is turned up, and the cutout pieces 24 are easily formed. As a result, the advantage of the cutout piece 24 as described above is obtained. In particular, the cutout piece 24 is suitably formed by the stress applied when the cutout piece 24 is stored in a container or the like without a step of positively forming the cutout piece 24 by a method described later.

As illustrated in FIG. 6, the small piece 1 may have a non-planar shape.

As a result, the pulp fiber inside the pulp fiber base material 2 can be more appropriately exposed, and the absorption rate of the liquid can be further increased.

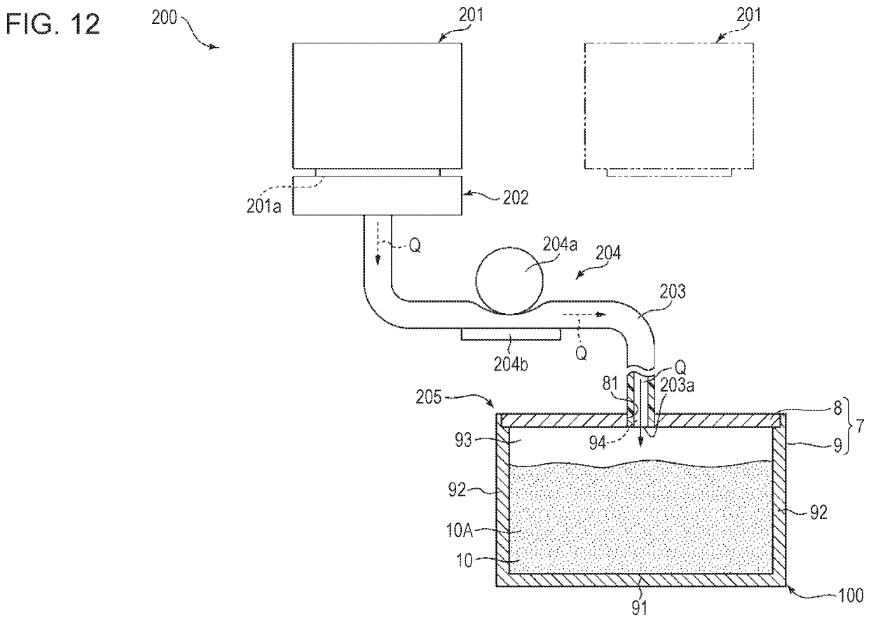

In addition, an appropriate gap can be formed between the small pieces 1, and the bulk density of the liquid absorbent body 10A can be adjusted more easily. In addition, the shape of the liquid absorbent body 10A can be changed more freely. Therefore, for example, as illustrated in FIG. 12 referred to in the description to be described later, a desired amount of the liquid absorbent body 10A can be suitably stored in the container, and it is possible to effectively prevent the occurrence of unintended unevenness in the absorption characteristics of the liquid at each portion in the container. Therefore, the absorption efficiency of the water-containing liquid can be made particularly excellent in the liquid absorbent body 10A in the state of being stored in the container.

In such a stored state, a gap is likely to be formed between the small pieces 1. As a result, the liquid can pass through the gap, and if the gap is too small, the liquid can wet and spread by capillary phenomenon, that is, the permeability of the liquid can be ensured. As a result, the liquid flowing downward in the container is prevented from being blocked in the middle, and thus can permeate into the bottom portion of the container. As a result, each of the small pieces 1 can suitably absorb the liquid and hold the liquid.

When the small piece 1 has a non-planar shape, it is preferable to satisfy the following conditions.

That is, when the liquid absorbent body 10A includes the small piece aggregate 10 provided with the plurality of small pieces 1 with a shape having a longitudinal direction, in other words, a longitudinal shape, the total length in the longitudinal direction of the small piece 1 is L1 [mm], and the distance between end points coupling one end and the other end of the small piece is L2 [mm], the average value of L2/L1 is preferably larger than 0 and 0.950 or less, more preferably 0.150 or more and 0.945 or less, still more preferably 0.300 or more and 0.940 or less, and most preferably 0.500 or more and 0.930 or less.

As a result, the above-described effects are more remarkably exhibited.

As illustrated in FIG. 6, the total length L1 in the longitudinal direction of the small piece 1 is a length of a line segment coupling one end and the other end of the small piece 1 in the longitudinal direction along the small piece 1, that is, a length in the longitudinal direction when the small piece 1 is curved or bent and is corrected to be planar.

In addition, the distance L2 between end points coupling one end and the other end of the small piece 1 is a length of a line segment coupling the one end and the other end of the small piece 1 with the shortest distance.

The dimensions of the small piece 1 can be measured, for example, using a digital microscope such as VHX-5000 manufactured by Keyence Corporation.

As the total length L1, for example, a middle point of each small piece 1 in the thickness direction can be measured at multiple points for each deformation point, and a length of multiple point coupling can be adopted.

As the distance L2 between the end points, for example, the distance between the two points when measuring two points at one end and the other end of the small piece 1 such as points S and T in FIG. 6 can be adopted.

As L2/L1 is closer to 1, it can be said that the small piece 1 is planar. The L1 and the L2 satisfy the relationship of L2≤L1, the L1 is a positive value, and the minimum value of L2 is zero. When one end and the other end of the small piece 1 is in contact with each other, and the small piece 1 is folded or has a ring shape, the L2=0. Therefore, L2/L1 can be rephrased as the planarity of the small piece 1, the larger the value of L2/L1, the greater the planarity, and the smaller the value of L2/L1, the smaller the planarity, that is, the greater the degree of deformation.

As the average value of L2/L1, the total length L1 in the longitudinal direction and the distance L2 between the end points are respectively measured for two or more predetermined numbers of small pieces 1 randomly extracted from the liquid absorbent body 10A, L2/L1 is determined, and the average value thereof is adopted.

Specifically, for example, two or more predetermined numbers, for example, sixteen small pieces 1 are randomly extracted from the liquid absorbent body 10A, L2/L1 for the extracted small pieces 1 are determined, and the average value thereof can be adopted as the average value of L2/L1.

In addition, for example, the liquid absorbent body 10A including the plurality of small pieces 1 is photographed using a charge coupled device (CCD) camera, a microscope or the like to obtain L2/L1 for the plurality of small pieces 1 included in a visual field range, and the average value thereof can be adopted as the average value of L2/L1.

The small piece 1 constituting the liquid absorbent body 10A may have a curved shape, a bent shape, a twisted shape, or a spiral shape. In addition, the small piece 1 may have a combination thereof.

As a result, in the small piece aggregate 10 constituting the liquid absorbent body 10A, the gap is suitably ensured between the small pieces 1 and it is possible to more easily adjust the bulk density of the liquid absorbent body 10A. In addition, the shape of the liquid absorbent body 10A can be changed more freely. Therefore, the desired amount of the liquid absorbent body 10A can be more suitably stored in the container, and the occurrence of unevenness in the absorption characteristics of the liquid can be more effectively prevented.

The method of forming the cutout portion 23 in the pulp fiber base material 2 can be selected according to the form of the cutout portion 23. For example, when the cutout portion 23 to be formed is a slit, the cutout portion 23 can be suitably formed using a slit cutter or the like. In addition, when the cutout portion 23 to be formed is a through-hole, the cutout portion 23 can be suitably formed using various cutters such as a hole punch, a die cutter, and various needles. In this case, as the needle, for example, an awl or a pin-holder may be used. In particular, when the cutout portion 23 to be formed has a form as illustrated in FIG. 4, as illustrated in FIG. 7, a through-hole as the cutout portion 23 can be suitably formed by passing a sheet material 2′ containing the pulp fiber between a punching roller 500 as a processing roller provided with a plurality of needles 501 as a plurality of cutout portion forming units on the peripheral surface, and a support roller 600. The peripheral surface of the support roller 600 may be made of a rubber material or the like. As a result, deterioration of the needle 501 and the support roller 600 can be suppressed. In addition, by setting the form of the cutout portion forming unit of the processing roller, for example, a cutout portion 23 other than the form illustrated in FIG. 4 can be suitably formed.

The small piece 1 includes the pulp fiber base material 2 containing the pulp fiber, and the water absorbent resin 3 carried on at least one surface side of the pulp fiber base material 2.

In the configuration illustrated in FIG. 8, although the water absorbent resin 3 is carried only on one surface side of the pulp fiber base material 2, the water absorbent resin 3 may be carried on both sides of the pulp fiber base material 2, that is, on a surface 21 on the front side and on a surface 22 on the rear side. In this case, it is preferable that the adhesion amount of the water absorbent resin 3 may differ between the surface 21 on the front side and the surface 22 on the rear side. As a result, the absorption and propagation of the liquid can be adjusted more suitably. The same applies to the configuration illustrated in FIG. 9.

### 1-1. Pulp Fiber Base Material

The pulp fiber base material 2 contains the pulp fiber, and is a support body for supporting the water absorbent resin 3 on the surface thereof. The water absorbent resin 3 can be suitably carried on the pulp fiber base material 2, and the detachment of the water absorbent resin 3 from the pulp fiber base material 2 can be more suitably prevented. In addition, when the liquid is applied to the small piece 1, the pulp fiber base material 2 temporarily holds the liquid, and thereafter the water absorbent resin 3 can be efficiently fed, and the absorption characteristics of the liquid of the entire small piece 1 can be improved. In addition, in general, pulp fibers are inexpensive than the water absorbent resin 3 and are also advantageous from the viewpoint of reducing the production cost of the small piece 1. In particular, when fibers derived from a waste paper are used as the pulp fibers, the above effects are more significantly exhibited. In addition, it is also advantageous from the viewpoint of waste reduction and effective use of resources.

The pulp fibers are mainly formed of cellulose. Since cellulose is a material having a suitable hydrophilic property, when a liquid is applied to the small piece 1, the liquid can be suitably taken in, a state of a particularly high fluidity, for example, of a viscosity of 10 mPa·s or less can be rapidly released, and the liquid once taken in can be suitably fed into the water absorbent resin 3. As a result, it is possible to make the absorption characteristics of the liquid of the entire small piece 1 particularly excellent. In addition, since cellulose has normally a high affinity to the water absorbent resin 3, the water absorbent resin 3 can be more suitably carried on the surface of the pulp fiber. In addition, the pulp fiber is a renewable natural material, and among various types of fibers, it is inexpensive and easily available, so that it is also advantageous from the viewpoints of reduction of production cost of small piece 1, stable production, reduction of environmental load, and the like.

In the present specification, the pulp fiber may be a fibrous material containing cellulose as a main component as a compound, and may include hemicellulose and lignin in addition to cellulose.

The lower limit of the average length of the pulp fibers is not particularly limited, and is preferably 0.1 mm.

The upper limit of the average length of the pulp fibers is not particularly limited, is preferably 7 mm, more preferably 5 mm, and still more preferably 3 mm.

The lower limit of the average width of the pulp fibers is not particularly limited, and is preferably 0.05 mm, and more preferably 0.1 mm.

The upper limit of the average width of the pulp fibers is not particularly limited, is preferably 2 mm, and more preferably 1 mm.

The average aspect ratio, that is, the lower limit of a ratio of average length to average width of the pulp fibers is not particularly limited, is preferably 10, and more preferably 15.

The upper limit of the average aspect ratio of the pulp fibers is not particularly limited, is preferably 1,000, and more preferably 500.

According to the above numerical range, it is possible to more suitably carry on the water absorbent resin 3, hold the liquid by the pulp fibers, and feed the liquid into the water absorbent resin 3, and it is possible to make the absorption characteristics of the liquid of the entire small piece 1 more excellent.

The pulp fiber base material 2 may include, for example, components other than the pulp fiber. Examples of such components include sizing agents and the like.

The sizing agent is a component used to prevent water and ink from bleeding into the paper and to apply water resistance. The sizing agent is used in various types of paper.

As will be described in detail later, even when the liquid absorbent body was configured to include an aggregate of small pieces having a pulp fiber base material that carries a water absorbent resin, even in a case in which the small piece was not provided with a cutout portion, if the pulp fiber base material contained a sizing agent, the liquid absorption efficiency was likely to be particularly low. On the other hand, in the present disclosure, even when the pulp fiber base material 2 constituting the small piece 1 contains the sizing agent, the liquid absorption efficiency can be made excellent.

Therefore, when the pulp fiber base material 2 contains the sizing agent, the effect of the present disclosure is more remarkably exhibited. In addition, in the present disclosure, various papers containing a sizing agent such as waste paper can be suitably used as the pulp fiber base material 2.

### 1-2. Water Absorbent Resin

The water absorbent resin 3 which is a component of the liquid absorbent body 10A may be any resin having a water absorbency and is not particularly limited. Examples thereof include carboxymethyl cellulose, polyacrylic acid, polyacrylamide, starch-acrylic acid graft copolymer, hydrolyzate of starch-acrylonitrile graft copolymer, vinyl acetate-acrylic ester copolymer, copolymer of isobutylene and maleic acid, hydrolyzate of acrylonitrile copolymer or acrylamide copolymer, polyethylene oxide, polysulfonic acid compound, polyglutamic acid, salts thereof, crosslinked products thereof, and the like. Here, the water absorbency refers to the function of having hydrophilicity and holding water. Many of the water absorbent resins 3 gel when absorbed by water.

Among these, the water absorbent resin 3 is preferably a resin having a functional group in the side chain. Examples of the functional group include an acid group, a hydroxyl group, an epoxy group, an amino group, and the like.

In particular, the water absorbent resin 3 is preferably a resin having an acid group in the side chain, and more preferably a resin having a carboxyl group in the side chain.

Examples of the carboxyl group-containing unit constituting the water absorbent resin 3 include acrylic acid, methacrylic acid, itaconic acid, maleic acid, crotonic acid, fumaric acid, sorbic acid, cinnamic acid, and those derived from monomers such as anhydrides and salts thereof.

When the liquid absorbent body 10A includes the water absorbent resin 3 having an acid group in the side chain, the lower limit of the proportion of acid groups contained in the water absorbent resin 3 which are neutralized to form a salt is preferably 30 mol %, more preferably 50 mol %, still more preferably 60 mol %, and most preferably 70 mol %.

When the liquid absorbent body 10A includes the water absorbent resin 3 having an acid group in the side chain, the upper limit of the proportion of acid groups contained in the water absorbent resin 3 which are neutralized to form a salt is preferably 100 mol %, more preferably 95 mol %, still more preferably 90 mol %, and most preferably 80 mol %.

When the proportion is as described above, a liquid absorbing property by the liquid absorbent body 10A can be made more excellent.

The type of the salt for neutralization is not particularly limited, examples thereof include alkali metal salts such as sodium salts, potassium salts and lithium salts, salts of nitrogen-containing basic substances such as ammonia, and the like, and sodium salts are preferred.

As a result, a liquid absorbing property by the liquid absorbent body 10A can be made more excellent.

The water absorbent resin 3 having an acid group in the side chain is preferable because electrostatic repulsion between the acid groups occurs at the time of liquid absorption, and an absorption rate is fast. In addition, when the acid group is neutralized, the liquid is likely to be absorbed inside the water absorbent resin due to osmotic pressure.

The water absorbent resin 3 may have a structural unit not containing an acid group, and examples of the structural unit include a hydrophilic structural unit, a hydrophobic structural unit, and structural unit containing a polymerizable crosslinking agent, and the like.

Examples of the hydrophilic structural unit include structural units derived from nonionic compounds such as acrylamide, methacrylamide, N-ethyl (meth) acrylamide, N-n-propyl (meth) acrylamide, N-isopropyl (meth) acrylamide, N, N-dimethyl (meth) acrylamide, 2-hydroxyethyl (meth) acrylate, 2-hydroxypropyl (meth) acrylate, methoxypolyethylene glycol (meth) acrylate, polyethylene glycol mono (meth) acrylate, N-vinyl pyrrolidone, N-acryloyl piperidine, and N-acryloyl pyrrolidine.

Examples of the hydrophobic structural unit include structural units derived from compounds such as (meth) acrylonitrile, styrene, vinyl chloride, butadiene, isobutene, ethylene, propylene, stearyl (meth) acrylate, and lauryl (meth) acrylate.

Examples of the structural unit to be the polymerizable crosslinking agent include structural units derived from diethylene glycol diacrylate, N, N′-methylene bisacrylamide, polyethylene glycol diacrylate, polypropylene glycol diacrylate, trimethylolpropane diallyl ether, trimethylolpropane triacrylate, allyl glycidyl ether, pentaerythritol triallyl ether, pentaerythritol diacrylate monostearate, bisphenol diacrylate, isocyanuric acid diacrylate, tetraallyloxyethane, and diallyloxyacetate.

As the water absorbent resin 3, a polyacrylate copolymer or a polyacrylic acid polymer crosslinked product is preferable from the viewpoint of absorption characteristics, cost and the like.

As a polyacrylic acid polymerization crosslinked product, the proportion of a structural unit having a carboxyl group in the total structural units constituting a molecular chain is preferably 50 mol % or more, more preferably 80 mol % or more, and still more preferably 90 mol % or more.

When the proportion of the structural unit containing a carboxyl group is too low, it may be difficult to make the absorption characteristics of the liquid sufficiently excellent.

The carboxyl group in the polyacrylic acid polymerization crosslinked product is preferably partially neutralized to form a salt.

The lower limit of the proportion of neutralized ones in the total carboxyl groups in the polyacrylic acid polymer crosslinked product is preferably 30 mol %, more preferably 50 mol %, and still more preferably 70 mol %.

It is preferable that the upper limit of the proportion of neutralized ones in all carboxyl groups in the crosslinked polyacrylic acid polymer is 99 mol %.

In addition, the water absorbent resin 3 may have a structure crosslinked by a crosslinking agent other than the above-described polymerizable crosslinking agent.

When the water absorbent resin 3 is a resin having an acid group, for example, a compound having a plurality of functional groups reactive with the acid group can be preferably used as the crosslinking agent.

When the water absorbent resin 3 is a resin having a functional group reactive with the acid group, a compound having a plurality of functional groups reactive with the acid group in the molecule can be suitably used as the crosslinking agent.

Examples of the crosslinking agent which is a compound having the plurality of functional groups reactive with the acid group include glycidyl ether compounds such as ethylene glycol diglycidyl ether, trimethylolpropane triglycidyl ether, (poly) glycerin polyglycidyl ether, diglycerin polyglycidyl ether, and propylene glycol diglycidyl ether; polyhydric alcohols such as (poly) glycerin, (poly) ethylene glycol, propylene glycol, 1,3-propanediol, polyoxyethylene glycol, triethylene glycol, tetraethylene glycol, diethanolamine, and triethanolamine; polyvalent amines such as ethylenediamine, diethylenediamine, polyethyleneimine, and hexamethylenediamine. In addition, polyvalent ions such as zinc, calcium, magnesium, and aluminum can be suitably used because these react with the acid groups of the water absorbent resin 3 to function as a crosslinking agent.

The water absorbent resin 3 may have any shape, for example, scaly, acicular, fibrous, or particulate shape, and the majority thereof is preferably in the form of particles. When the water absorbent resin 3 is in the form of particles, the permeability of the liquid can be easily ensured. In addition, the water absorbent resin 3 can be suitably carried on the pulp fiber base material 2. The particulate shape refers to the ratio of the maximum length to the minimum length is 0.7 or more and 1.0 or less.

The lower limit of the average particle diameter of the particles is preferably 10 μm, more preferably 20 μm, and still more preferably 30 μm.

The upper limit of the average particle diameter of the particles is preferably 800 μm, more preferably 600 μm, and still more preferably 500 μm.

When the average particle diameter is as described above, the above-described effects can be more reliably exerted.

On the other hand, when the average particle diameter of the particles is too small, the permeability of the liquid to the inside of the liquid absorbent body 10A is likely to be reduced.

In addition, when the average particle diameter of the particles is too large, a specific surface area of the water absorbent resin 3 is small, the absorption characteristics of the liquid decrease, and the absorption rate of the liquid decreases.

In the present disclosure, the average particle diameter refers to a volume-based average particle diameter. The average particle diameter can be determined, for example, by measurement with a particle diameter distribution measuring device having a laser diffraction and scattering method as a measurement principle, that is, a laser diffraction type particle diameter distribution measuring device.

In addition, in a case in which the water absorbent resin 3 is in the form of particles, when the average particle diameter of the water absorbent resin 3 is D [μm] and the average length of the pulp fiber is L [μm], it is preferable to satisfy the relationship of 0.15≤L/D≤467, more preferable to satisfy the relationship of 0.25≤L/D≤333, and still more preferable to satisfy the relationship of 2≤L/D≤200.

As a result, it is possible to more suitably carry on the water absorbent resin 3, hold the liquid by the pulp fibers, and feed the liquid into the water absorbent resin 3, and it is possible to make the absorption characteristics of the liquid of the entire liquid absorbent body 10A more excellent.

The particles may contain components other than the water absorbent resin. Examples of such components include surfactants, lubricants, antifoaming agents, fillers, anti-blocking agents, ultraviolet absorbing agents, and the like.

The water absorbent resin 3 may have a uniform structure as a whole, or may have a different structure at each portion. For example, in the water absorbent resin 3, the area near the surface, more specifically, the area with a thickness of 1 μm from the surface may have a higher degree of cross-linking than that of other portion.

As a result, it is possible to improve an absorption ratio and an absorption rate of the liquid, strength of the water absorbent resin 3, and the like in a more balanced manner.

In addition, the adhesion between the water absorbent resin 3 and the pulp fibers can be made more excellent, the liquid once held by the pulp fibers can be fed efficiently by the water absorbent resin, and the absorption characteristics of the entire liquid absorbent body 10A can be further improved.

In the configuration illustrated in FIG. 8, the water absorbent resin 3 is carried on one surface side of the pulp fiber base material 2. In addition, a portion of the water absorbent resin 3 is penetrated inward from one surface of the pulp fiber base material 2. That is, a portion of the water absorbent resin 3 is impregnated in the pulp fiber base material 2. As a result, the carrying capacity of the water absorbent resin 3 to the pulp fiber base material 2 can be enhanced. Therefore, the water absorbent resin 3 can be prevented from detaching in the container. As a result, the high absorption characteristics of the liquid can be exhibited over a long period of time, the water absorbent resin 3 can be prevented from being unevenly distributed in the container, and the occurrence of unevenness in the absorption characteristics of the liquid can be prevented.

In the present specification, “impregnation” refers to a state of being embedded in which at least a portion of the particles of the water absorbent resin 3 penetrates inward from the surface of the pulp fiber base material 2. In addition, it is not necessary for all particles to be impregnated. In addition, a state where the particles of the water absorbent resin 3 penetrate in the inside of the pulp fiber base material 2 by softening and come out to the rear surface of the pulp fiber base material 2 is also included.

In addition, in the configuration illustrated in FIG. 9, the small piece 1 has a pair, that is, two pulp fiber base materials 2. The water absorbent resin 3 is provided between these pulp fiber base materials 2. In other words, in the present embodiment, the small pieces 1 have a plurality of stacked pulp fiber base materials 2, and the water absorbent resin 3 is provided between each of the pulp fiber base materials 2.

As a result, the water absorbent resin 3 has a configuration in which each of the pulp fiber base materials 2 is interposed and covered, and is prevented from being exposed to the outer surface of the small piece 1. As a result, the water absorbent resin 3 is more effectively prevented from detaching from the pulp fiber base material 2. Therefore, the high absorption characteristics of the liquid can be exhibited over a longer period of time, the water absorbent resin 3 can be more effectively prevented from being unevenly distributed in the container, and the occurrence of unevenness in the absorption characteristics of the liquid can be more effectively prevented.

When the small piece 1 has a structure in which the water absorbent resin 3 is disposed between the pair of pulp fiber base materials 2, it is preferable that at least one of the pair of pulp fiber base materials 2 constituting the small piece 1 is provided with the cutout portion 23 penetrating the pulp fiber base material 2 in the thickness direction.

As a result, as will be described in detail later, it is possible to effectively utilize many portions of the pulp fiber base material 2 provided with the cutout portion 23 in the thickness direction, and to efficiently absorb a large amount of the liquid in a short time. In addition, the liquid permeated through the cutout portion 23 can be efficiently supplied to the water absorbent resin 3 and the pulp fiber base material 2 on the opposite side. In addition, the liquid absorption efficiency of the small pieces 1 can be further improved.

In particular, it is preferable that cutout portions 23 are provided in corresponding portions of the pair of pulp fiber base materials 2 constituting the small pieces 1 as through-holes penetrating the pulp fiber base material 2 in the thickness direction.

As a result, the liquid permeated from the cutout portion 23 provided in one pulp fiber base material 2 can be efficiently supplied to the cutout portion 23 of the other pulp fiber base material 2. It is possible to effectively utilize many portions of both the pulp fiber base material 2 in the thickness direction, and to efficiently absorb a large amount of the liquid in a short time.

In the illustrated configuration, the small piece 1 has two pulp fiber base materials 2 and the water absorbent resin 3 is disposed between these pulp fiber base materials 2. For example, the small piece 1 may have three or more pulp fiber base materials 2, and the water absorbent resin 3 may be disposed between these each of the pulp fiber base materials 2.

In the configuration illustrated in FIGS. 8 and 9, the small piece 1 has a sizing agent layer 25 containing a sizing agent at a higher content than that of other portions of the small piece 1.

As described above, the sizing agent is a component used to prevent water or ink from bleeding into paper and to apply water resistance. The sizing agent is used in various types of paper.

Here, referring to FIGS. 10 and 11, for the small piece having the cutout portion and the small piece having no cutout portion, when the liquid is applied from one main surface side of the small piece, the absorption of the liquid by the small piece will be described. In FIGS. 10 and 11, illustration of the water absorbent resin 3 is omitted.

As illustrated in FIG. 10, in a case where in which the pulp fiber base material 2 does not have the cutout portion 23, if the small piece 1 has the sizing agent layer 25 containing a sizing agent at a higher content than that of other portions, when a liquid L is applied to the small piece 1 from the one main surface side, the liquid L reaching the sizing agent layer 25 is inhibited by the sizing agent layer 25 from permeating in the thickness direction beyond the sizing agent layer 25. As a result, even when the liquid L can be efficiently absorbed on one main surface side of the small piece 1, the liquid L cannot be sufficiently absorbed on the other surface side.

On the other hand, as illustrated in FIG. 11, when the pulp fiber base material 2 has the cutout portion 23, even if the small piece 1 has the sizing agent layer 25 containing the sizing agent at a higher content than that of other portions, the liquid L can be efficiently absorbed on both sides of the small piece 1. That is, when the liquid L is applied to the small piece 1 from one main surface side, the liquid L can appropriately permeate into the inside of the pulp fiber base material 2 through the cutout portion 23 regardless of the presence of the sizing agent layer 25. The liquid L is suitably absorbed by the pulp fiber base material 2 from the inner wall surface of the cutout portion 23 reaching a deeper portion than the sizing agent layer 25. Therefore, when the pulp fiber base material 2 has the cutout portion 23, regardless of the presence or absence of the sizing agent layer 25, it is possible to effectively utilize many portions of the small piece 1 in the thickness direction, and to efficiently absorb a large amount of the liquid in a short time. Here, examples of the liquid L include an ink Q described later.

In FIG. 11, the cutout portion 23 is a through-hole, and the same effect can be obtained when the cutout portion 23 is a slit. In addition, in FIG. 11, the small piece 1 is provided with only one pulp fiber base material 2, and as illustrated in FIG. 9, even when the small piece 1 is provided with a plurality of pulp fiber base materials 2, the same effect can be obtained.

The lower limit of the ratio of the content of the water absorbent resin 3 to the content of the pulp fibers in the small piece 1 is preferably 25% by mass, and more preferably 50% by mass.

The upper limit of the ratio of the content of the water absorbent resin 3 to the content of the pulp fibers in the small piece 1 is preferably 300% by mass, and more preferably 150% by mass.

With the above ratio, water absorbency and permeability can be sufficiently ensured.

When the content of the water absorbent resin 3 in the small pieces 1 is too low, the water absorbency may be insufficient. On the other hand, when the content of the water absorbent resin 3 in the small piece 1 is too high, there is a possibility that the expansion coefficient of the small piece 1 tends to increase, and the permeability may be reduced.

In addition, the small pieces 1 may contain components other than those described above.

Examples of such components include surfactants, lubricants, antifoaming agents, fillers, anti-blocking agents, ultraviolet absorbing agents, colorants such as pigments and dyes, flame retardants, flow improvers, and the like.

The content of the other components in the small piece 1 is preferably 10% by mass or less, and more preferably 5.0% by mass or less.

The preferred range of the length in the longitudinal direction of the small piece 1 is not particularly limited, and depends on the shape and size of the container. The lower limit of the average value of the length in the longitudinal direction of the small piece 1 is preferably 0.5 mm, more preferably 1.0 mm, and still more preferably 2.0 mm. The upper limit of the average value of the length in the longitudinal direction of the small piece 1 is preferably 200 mm, more preferably 100 mm, and still more preferably 30 mm.

With the above length, it is possible to more suitably carry on the water absorbent resin 3, hold the liquid by the pulp fibers, and feed the liquid into the water absorbent resin 3, and it is possible to make the absorption characteristics of the liquid of the entire small piece 1 more excellent.

In addition, the preferred range of the width of the small piece 1 is not particularly limited, and depends on the shape and size of the container. The lower limit of the average value of the width of the small pieces 1 is preferably 0.1 mm, more preferably 0.3 mm, and still more preferably 1 mm. The upper limit of the average value of the width of the small pieces 1 is preferably 100 mm, more preferably 50 mm, and still more preferably 20 mm.

In addition, the lower limit of the average value of the aspect ratio, which is the ratio of the length to the width for each small piece 1, is preferably 2.0, and more preferably 2.5. The upper limit of the average value of the average aspect ratio, which is the ratio of the length to the width for each small piece 1, is preferably 200, and more preferably 30.

The lower limit of the thickness of the small piece 1 is preferably 0.05 mm, and more preferably 0.1 mm. The upper limit of the thickness of the small piece 1 is preferably 3 mm, more preferably 2 mm.

According to the above numerical range, it is possible to more suitably carry on the water absorbent resin 3, hold the liquid by the pulp fibers, and feed the liquid into the water absorbent resin 3, and it is possible to make the absorption characteristics of the liquid of the entire small piece 1 more excellent. Furthermore, the entire liquid absorbent body 10A is likely to be deformed, and the shape followability to the container is excellent.

The liquid absorbent body 10A may include small pieces 1 having different sizes and shapes.

In addition, the liquid absorbent body 10A may include the small pieces 1 in which at least one of the length, width, aspect ratio, and thickness are the same as each other, or may include the small pieces 1 in which all of these are different from each other.

The lower limit of the content of the small pieces 1 having a maximum width of 3 mm or less in the liquid absorbent body 10A is preferably 30% by mass, and more preferably 40% by mass. The upper limit of the content of the small pieces 1 having a maximum width of 3 mm or less in the liquid absorbent body 10A is preferably 90% by mass, and more preferably 80% by mass. With the above content, the occurrence of unevenness in the absorption characteristics of the liquid can be more effectively prevented.

If the content of the small piece 1 having a maximum width of 3 mm or less is too low, when the liquid absorbent body 10A is stored in the container, a gap is likely to be formed between the small pieces 1 and there is a concern that unevenness occurs in the absorption characteristics of the liquid in the container. On the other hand, when the content of the small piece 1 having a maximum width of 3 mm or less is too high, it tends to be unlikely to form a gap between the small pieces 1.

In addition, the shape of the small piece aggregate 10 can be freely changed. Therefore, the liquid absorbent body 10A can be appropriately stored in a container in a desired amount, and it is possible to effectively prevent unevenness in the liquid absorption characteristics at each portion in the container.

In addition, since each of the small pieces 1 is stored at random, the entire liquid absorbent body 10A has an increased chance of contacting the liquid, and thus the absorption performance absorbing the liquid is improved. In addition, when the liquid absorbent body 10A is stored in the container, each of the small pieces 1 can be randomly put into the container, and thus the storing operation can be performed easily and rapidly.

In addition, the lower limit of the bulk density of the liquid absorbent body 10A is preferably 0.01 g/cm³, more preferably 0.05 g/cm³, and still more preferably 0.08 g/cm³. In addition, the upper limit of the bulk density of the liquid absorbent body 10A is preferably 0.50 g/cm³, more preferably 0.30 g/cm³, and still more preferably 0.25 g/cm³.

With the above bulk density, the liquid holding power and the permeability of the liquid absorbent body 10A can be compatible at a higher level.

The plurality of small pieces 1 constituting the liquid absorbent body 10A may have the same condition for the layer configuration and the cutout portion 23 as each other, or may have at least one condition different from each other.

The liquid absorbent body 10A is used by being stored in a predetermined container, for example, by measuring a desired amount, and manual loosening.

The number of pieces of the small pieces 1 stored in the container is not particularly limited, and for example, the necessary number of pieces may be appropriately selected according to various conditions such as the use of the liquid absorbent body 10A. The maximum absorption amount of the liquid in the liquid absorbent body 10A is adjusted depending on the size of the storage amount of the small pieces 1.

In addition, the liquid absorbent body 10A may include a configuration other than the small piece 1. For example, a pulp fiber as a defibrated material, a water absorbent resin not carried on a pulp fiber base material 2, or a small piece of the pulp fiber not carrying the water absorbent resin may be included. In addition, in addition to the small piece 1 provided with the cutout portion 23 as described above, a small piece not provided with the cutout portion 23 may be included. However, the content of the components other than the small pieces 1 provided with the cutout portion 23 in the liquid absorbent body 10A is preferably 10% by mass or less, more preferably 5% by mass or less, and still more preferably 1% by mass or less.

### 2. Liquid Absorber and Liquid Treatment Apparatus

Next, a liquid absorber provided with the liquid absorbent body of the present disclosure and a liquid treatment apparatus will be described. In the following description, an ink absorber as an example of the liquid absorber and a printing apparatus as an example of the liquid treatment apparatus will be described.

FIG. 12 is a partial vertical cross-sectional view illustrating an example of an ink absorber as a liquid absorber using the liquid absorbent body as an ink absorbent material, and an example of a printing apparatus as a liquid treatment apparatus.

The liquid absorber according to the present disclosure is provided with the liquid absorbent body and the container that stores the liquid absorbent body.

The ink absorber 100 as the liquid absorber illustrated in FIG. 12 is provided with the liquid absorbent body 10A as the ink absorbent material, and the container 7 for storing the liquid absorbent body 10A. The container 7 includes the container main body 9 and the lid 8 that covers the upper opening 94 of the container main body 9.

The liquid treatment apparatus according to the present disclosure includes the liquid absorber, and the liquid absorber is provided with a recovery portion that recovers a waste liquid.

A printing apparatus 200 as the liquid treatment apparatus illustrated in FIG. 12 is, for example, an ink jet type color printer and is provided with a recovery portion 205 that recovers the waste liquid of the ink Q. The ink absorber 100 as the liquid absorber is provided in the recovery portion 205.

According to the ink absorber 100 and the printing apparatus 200 configured as described above, the shape of the entire liquid absorbent body 10A changes following the shape of the storage space 93 of the container 7, and it is possible to effectively prevent unintended variations in the density of the liquid absorbent body 10A from occurring in the container 7. As a result, the absorption efficiency of the water-containing liquid can be made excellent in the liquid absorbent body 10A in a state of being stored in the container. In addition, the cutout portion 23 that exposes the pulp fiber inside the pulp fiber base material 2 is provided at a portion other than the outer peripheral end surface of the pulp fiber base material 2 that defines the outer shape of the small piece 1. Therefore, the cutout portion 23 can also absorb the liquid in addition to the both main surfaces and the end surfaces of the small piece 1, and the absorption rate of the liquid of the liquid absorbent body 10A as a whole can be made excellent. In particular, in the cutout portion 23, normally, the capillary phenomenon is effectively exerted, so that the absorption rate of the liquid can be made more excellent.

In the present specification, “ink absorption” refers to absorbing the entire ink such as a solvent-based ink in which a binder is dissolved in a solvent, an UV curable ink in which a binder is dissolved in a liquid monomer which is cured by UV irradiation, and a latex ink in which a binder is dispersed in a dispersion medium, as well as absorbing an aqueous ink in which a coloring material is dissolved in an aqueous solvent. In particular, the present disclosure is preferably applied to an ink having a water content of 50% by mass or more.

The printing apparatus 200 includes an ink ejection head 201 ejecting the ink Q, a capping unit 202 preventing clogging of nozzles 201a of the ink ejection head 201, a tube 203 coupling the capping unit 202 and the ink absorber 100, a roller pump 204 transferring the ink Q from the capping unit 202, and the recovery portion 205.

The ink ejection head 201 has a plurality of nozzles 201a ejecting the ink Q downward. The ink ejection head 201 can eject the ink Q and perform printing while moving relative to a recording medium (not illustrated) such as a PPC sheet.

The capping unit 202 collectively sucks each of the nozzles 201a by the operation of the roller pump 204 when the ink ejection head 201 is in a standby position, and prevents clogging of the nozzles 201a.

The tube 203 is a tube passing the ink Q sucked through the capping unit 202 toward the ink absorber 100. The tube 203 is flexible.

The roller pump 204 is disposed in the middle of the tube 203, and includes a roller portion 204a and a pinching portion 204b which pinches the middle of the tube 203 between the pinching portion 204b and the roller portion 204a. The rotation of the roller portion 204a generates a suction force on the capping unit 202 through the tube 203. The roller portion 204a keeps rotating, so that the ink Q adhering to the nozzle 201a can be fed to the recovery portion 205.

The recovery portion 205 is provided with the ink absorber 100 storing the liquid absorbent body 10A used as the ink absorbent material. The ink Q passed through the tube 203 is sent to the ink absorber 100 and is absorbed as waste liquid by the liquid absorbent body 10A in the ink absorber 100. The ink Q contains ink of various colors.

As illustrated in FIG. 12, the ink absorber 100 is provided with the liquid absorbent body 10A, the container main body 9 that stores the liquid absorbent body 10A, and the lid 8 that covers the upper opening 94 of the container main body 9. The container 7 is configured to include the container main body 9 and the lid 8.

The ink absorber 100 is detachably attached to the printing apparatus 200, and in the attached state, is used to absorb the waste liquid of the ink Q as described above. As described above, the ink absorber 100 can be used as a so-called “waste liquid tank”. When the absorption amount of the ink Q of the ink absorber 100 reaches the limit, the ink absorber 100 can be replaced with a new ink absorber 100. A detection portion (not illustrated) in the printing apparatus 200 detects whether or not the absorption amount of the ink Q of the ink absorber 100 reaches the limit. In addition, when the absorption amount of the ink Q of the ink absorber 100 reaches the limit, that effect is notified by, for example, a notification portion such as a monitor incorporated in the printing apparatus 200.

The container 7 stores the liquid absorbent body 10A. The container main body 9 has a box shape including a bottom portion 91 formed of, for example, a bottom plate having a rectangular shape in plan view as viewed from above, and four side wall portions 92 erected upward from each side of the bottom portion 91. The liquid absorbent body 10A is stored in a predetermined portion in a storage space 93 surrounded by the bottom portion 91 and the four side wall portions 92. The lid 8 is a plate shaped member that covers the upper opening 94 of the container main body 9.

The container main body 9 is not limited to the one having the bottom portion 91 having a square shape in plan view, may have, for example, the bottom portion 91 having a circular shape in plan view, and the whole may be cylindrical.

The container 7 is hard, in other words, has a shape-retaining property such that the volume does not change by 10% or more when an internal pressure or an external force acts on the container 7. As a result, the container 7 can maintain the shape of the container 7 itself even when each of the small pieces 1 of the liquid absorbent body 10A absorbs the ink Q, and thereafter expands to receive the force from the small piece 1 from the inside. Therefore, the installation state of the container 7 in the printing apparatus 200 is stabilized, and each of the small pieces 1 can stably absorb the ink Q.

The container 7, especially the container main body 9, may be made of a material that does not transmit the ink Q, and although the constituent material is not particularly limited, various resin materials such as cyclic polyolefin and polycarbonate can be used, for example. In addition, as the constituent material of the container 7, various metal materials such as aluminum and stainless steel can be used in addition to the various resin materials, for example.

In addition, the container 7, especially the container main body 9, may be transparent with internal visibility or opaque. Here, “transparent” is a concept including translucency, as long as it has visibility to the extent that an outline of the liquid absorbent body 10A inside the container 7, or a portion to which the ink Q of the liquid absorbent body 10A is attached can be identified.

As described above, the ink absorber 100 is provided with the lid 8 that covers the upper opening 94 of the container main body 9. As illustrated in FIG. 12, the lid 8 has a plate shape and can be fitted to the container main body 9 in a liquid-tight manner. As a result, for example, when the ink Q is discharged from the tube 203 and dropped, even in a case in which the ink Q collides with the liquid absorbent body 10A and jumps up, the ink Q can be prevented from scattering outward. Therefore, the ink Q can be prevented from adhering to the periphery of the ink absorber 100 and being soiled.

A coupling port 81 to which the tube 203 is coupled is formed at a central portion of the lid 8. The coupling port 81 is configured to include a through-hole which penetrates the lid 8 in the thickness direction. The downstream end portion of the tube 203 can be inserted into and coupled to the coupling port 81 in a liquid-tight manner. In addition, at this time, a discharge port 203a of the tube 203 faces downward.

For example, radial ribs or grooves may be formed in the vicinity of the coupling port 81 on the lower surface of the lid 8. The rib or the groove can function as, for example, a regulation portion that regulates the flow direction of the ink Q in the container 7.

In addition, the lid 8 may have an absorbing property to absorb the ink Q, or may have a lyophobic property to repel the ink Q.

The lower limit of the thickness of the lid 8 is not particularly limited, and is, for example, preferably 1 mm, and more preferably 8 mm. In addition, the upper limit of the thickness of the lid 8 is not particularly limited, and is, for example, preferably 20 mm, and more preferably 10 mm. The lid 8 is not limited to a plate shape having such a numerical range, and may be a film-like one thinner than the plate shape. In this case, the thickness of the lid 8 is not particularly limited, and is preferably, for example, 10 μm or more and less than 1 mm.

A vent portion (not illustrated) may be provided on the side surface of the container 7, for example, the lid 8 or the container main body 9.

The number of pieces of the small pieces 1 stored in the container 7 is not particularly limited, and the necessary number may be appropriately selected according to various conditions such as the use of the ink absorber 100, for example. As described above, the ink absorber 100 has a simple configuration in which the required number of small pieces 1 is stored in the container 7. The maximum absorption amount of the ink Q in the ink absorber 100 is adjusted according to the size of the storage amount of the small pieces 1.

Hereinbefore, although the preferred embodiments of the present disclosure are described, the present disclosure is not limited to the above embodiments.

For example, in the above-described embodiment, although the plurality of cutout portions are provided in a single small piece, the number of cutout portions provided in the small piece is not particularly limited, and the small piece may have at least one cutout portion.

In addition, in the above-described embodiment, the plurality of cutout portions provided in a single small piece are all of the same form, and the cutout portions of different forms may coexist in the single small piece. For example, among the through-hole and the slit penetrating the pulp fiber base material in the thickness direction, two or more cutout portions may coexist in the single small piece. In addition, when the single small piece has a plurality of slits as the cutout portions, the lengths thereof may be the same as each other or at least one length may be different from each other. In addition, when the single small piece has the plurality of slits as the cutout portions, the extending directions thereof may be the same as each other, or at least one extending direction may be different from each other.

In addition, in the above-described embodiment, the case where the cutout piece is formed in the pulp fiber base material and the case where the cutout piece is formed only on one surface side of the pulp fiber base material are described, the cutout piece may be formed on both surface sides of the pulp fiber base material.

In addition, the pulp fiber base material may have a cutout portion such as a through-hole reaching the outer peripheral end surface, in addition to the cutout portions provided other than the outer peripheral end surface that defines the outer shape of the small piece.

In addition, in the above-described embodiment, although the case where the small piece constituting the liquid absorbent body carries on the water absorbent resin on the surface of the pulp fiber base material is described, the small piece constituting the liquid absorbent body may uniformly contain the fiber and the water absorbent resin in each portion.

In addition, in the above-described embodiment, although the ink absorber is typically described as an example of the liquid absorber, and the printing apparatus is typically described as an example of the liquid treatment apparatus, the liquid absorber and the liquid treatment apparatus are not limited to these. For example, the liquid absorber may be an absorber that absorbs liquid other than ink. In addition, the liquid treatment apparatus may be, for example, various types of liquid application apparatuses such as a spray type and a dispense type, and a liquid recovery apparatus.

In addition, the liquid absorbent body of the present disclosure can be used, for example, as a liquid absorbent body in a waste liquid portion of a suction system for microbiology and cell culture.

In addition, the liquid absorbent body of the present disclosure can be used, for example, as an absorbent body for water or a water-soluble solution at the time of disaster or accident. The liquid absorbent body of the present disclosure absorbs a large amount of liquid, and can be discarded by suitably drying to reduce the weight when discarded. In addition, although disposal in a solution state requires enormous treatment and a large environmental load, treatment as a solid can be performed by applying the liquid absorbent body of the present disclosure.

What is claimed is:

1. A liquid absorbent body comprising:
a water absorbent resin; and
a small piece aggregate that includes a plurality of small pieces having a pulp fiber base material carrying the water absorbent resin, wherein
the pulp fiber base material is provided with a cutout portion that exposes a pulp fiber inside the pulp fiber base material, other than an outer peripheral end surface that defines an outer shape of the small piece.

2. The liquid absorbent body according to claim 1, wherein
the cutout portion is provided at a depth of half or more of the pulp fiber base material in a thickness direction.

3. The liquid absorbent body according to claim 1, wherein
the cutout portion is provided as a slit that penetrates the pulp fiber base material in a thickness direction.

4. The liquid absorbent body according to claim 1, wherein
the cutout portion is provided as a through-hole that penetrates the pulp fiber base material in a thickness direction.

5. The liquid absorbent body according to claim 3, wherein
the small piece includes a cutout piece formed by the cutout portion that penetrates the pulp fiber base material in the thickness direction, and is coupled to a periphery of the cutout portion.

6. The liquid absorbent body according to claim 1, wherein
the small piece includes a pair of pulp fiber base materials, and the water absorbent resin is provided between the pulp fiber base materials.

7. The liquid absorbent body according to claim 6, wherein
at least one of the pair of pulp fiber base materials that constitute the small piece is provided with the cutout portion that penetrates the pulp fiber base material in a thickness direction.

8. The liquid absorbent body according to claim 7, wherein
the cutout portion is provided as a through-hole that penetrates the pulp fiber base material in the thickness direction at a corresponding portion of the pair of pulp fiber base materials that constitute the small piece.

9. The liquid absorbent body according to claim 1, wherein
the cutout portion is coupled to an outer shape of the pulp fiber base material of the small piece.

10. The liquid absorbent body according to claim 1, wherein
the small piece has a non-planar shape.
